(19) United States
(12) Patent Application Publication
(10) Pub. No.: US 2021/0325386 A1
(43) Pub. Date: Oct. 21, 2021

(54) Microfluidic Devices and Methods for Rapid Detection of Pathogens and Other Analytes

(71) Applicant: University of Rochester, Rochester, NY (US)

(72) Inventor: James L. McGrath, Fairport, NY (US)

(73) Assignee: University of Rochester, Rochester, NY (US)

(21) Appl. No.: 17/176,135

(22) Filed: Feb. 15, 2021

Related U.S. Application Data
(60) Provisional application No. 63/076,893, filed on Sep. 10, 2020.
(60) Provisional application No. 63/010,675, filed on Apr. 15, 2020.

(51) Int. Cl. G01N 33/569; B01L 3/00; B01D 39/20

(52) U.S. Cl. CPC G01N 33/569; B82Y 30/00; B01D 39/2068; B01L 3/502746

(57) ABSTRACT

The invention provides devices and methods for detecting viruses, bacteria, and other analytes of interest in a fluid sample. The fluid sample flows through a first microfluidic channel to a nanoporous or microporous membrane on which are disposed ligands, such as antibodies, specific for the analyte. If the analyte of interest is captured by the ligand, it clogs the pores of the membrane, preventing the fluid sample from passing through the membrane and diverting the fluid into a second channel. Detecting movement of the fluid sample in the second channel signals the presence of the analyte in the fluid sample, while failure of the fluid sample to move in the second channel signals absence of the analyte in the fluid sample.

### CROSS-REFERENCE TO RELATED APPLICATIONS

This application claims priority to, and the benefit of, U.S. Provisional Patent Application No. 63/076,893, filed Sep. 10, 2020, and U.S. Provisional Patent Application No. 63/010,675, filed Apr. 15, 2020, the contents of each of which are incorporated herein by reference.

### STATEMENT OF FEDERAL FUNDING

This invention was made with government support under Grant IIP PFI-TT 1917902, awarded by the National Science Foundation. The government has certain rights in the invention.

### PARTIES TO JOINT RESEARCH AGREEMENT

Not applicable.

### BACKGROUND OF THE INVENTION

The current global pandemic caused by the novel corona virus SARS-CoV-2 has revealed the need for a rapid and inexpensive diagnostic test enabling population-based screening for infection. The circumstances surrounding testing for COVID-19 (the disease caused by SARS-CoV-2), are changing quickly. In March 2020, the United States expanded the national testing capacity from tens of thousands to millions of tests. Tests and results are now available through most hospitals and clinics including large commercial labs like Quest Diagnostics. Symptomatic patients are generally tested by RT-PCR and typically wait two to three days for test results to come back. On Mar. 27, 2020, Abbott Laboratories (Abbott Park, Ill.) announced the distribution of a ˜5-15 minute test based on its IDNow™ DNA-detection platform that is already in use for viral illnesses. However, the costs of point-of-care (POC) molecular diagnostic kits are high, and this will limit their use in low resource settings where COVID-19 is most devastating.

In contrast to molecular diagnostics, lateral flow immunological assays are fast, inexpensive, and don't require a separate unit to process and read. These assays detect the presence of antibodies in patient samples using the classic “sample-in, answer-out” format of a pregnancy test. Immunological test kits are now being offered by several companies Immunological assays, however, typically are only sensitive enough to permit detecting antibodies in a patient's blood approximately two weeks after exposure to a pathogen. While immunological assays are useful in detecting persons who have had a COVID-19 infection and cleared it, they are not helpful for detecting active infection.

It would be desirable to have additional methods to detect the presence of SARS-CoV-2 virus, of other viruses, of bacteria, of protozoa, or of cells in a patient sample. Surprisingly, the present invention fulfills these and other needs.

### SUMMARY OF THE INVENTION

The invention provides microfluidic devices and methods for rapidly determining if an analyte of interest is present in a fluid sample. In a first group of embodiments, the invention provides microfluidic devices for detecting an analyte of interest in a fluid sample, wherein the devices comprise: (a) a first microfluidic channel and a second microfluidic channel, each channel having a lumen defining a fluid path through the channel, the fluid path of each channel having a first end and a second end, wherein the first channel is positioned such that fluid sample will flow preferentially through the first channel in the absence of clogging of the fluid flow in the first channel, (b) a port for introducing the fluid sample into the first channel, the port being fluidly connected to the first end of the first channel, (c) a first nanoporous or microporous membrane disposed in the device such that the fluid sample must pass through the first membrane in the first channel to reach the second end of the first channel, and that, if the membrane becomes clogged, the fluid sample will be diverted instead into the first end of the second channel, (d) ligands that specifically or non-specifically bind the analyte of interest disposed on the first membrane, whereby binding of the analyte of interest to the membrane clogs the first membrane, (e) either (1) an indicator fluid or bubble disposed in second channel, wherein the indicator will move if the fluid sample is diverted into said second channel by said clogging of said membrane, or (2) wherein the second channel further has an exit port disposed at the second end of the second channel which allows fluid diverted into the first end of the second channel by blocking of the membrane to exit the channel, wherein movement of the indicator fluid or bubble, or exit of fluid from the exit port of the second channel, or bubbling at the exit port of the second channel, indicates the detection of the analyte of interest in the fluid sample. In some embodiments, the membrane is nanoporous. In some embodiments, the membrane is microporous. In some embodiments, the membrane is ultrathin. In some embodiments, the ultrathin membrane is made of silicon, silicon nitride, silicon oxide, or silicon dioxide. In some embodiments, the analyte of interest is a virus. In some embodiments, the virus is SARS-CoV-2. In some embodiments, the analyte is a bacterium. In some embodiments, the analyte of interest is a protozoan. In some embodiments, the fluid sample is from a patient. In some embodiments, the second channel contains fluid having an indicator fluid or a bubble. In some embodiments, the indicator fluid is an immiscible fluid. In some embodiments, the immiscible fluid is an oil. In some embodiments, the oil is colored. In some embodiments, the indicator is a bubble. In some embodiments, the ligand specifically binds the analyte. In some embodiments, the ligand is an antibody or antibody fragment retaining antigen-binding specificity. In some embodiments, the ligand non-specifically binds the analyte. In some embodiments, the ligand carries a charge that is opposite an overall charge on the analyte.

In a second group of embodiments, the invention provides methods for detecting whether an analyte of interest is present in a fluid sample, the analyte having a size, the methods comprising: (a) selecting a first nanoporous or microporous membrane with pore sizes larger than the size of the analyte, (b) purifying the fluid sample by flowing it through the nanoporous or microporous membrane to remove debris that is larger than the size of the analyte, thereby obtaining a purified fluid sample, (c) introducing the purified fluid sample to a port of a first channel of a microfluidic device, thereby flowing the purified fluid sample into the first channel, (d) contacting the purified fluid sample to a second nanoporous or microporous membrane, which membrane is disposed in said first channel, and which bears ligands that specifically or non-specifically bind the analyte of interest to the membrane, whereby binding of the analyte of interest to the membrane clogs the membrane and diverts the purified fluid sample into a second channel, and wherein if the analyte of interest is not present, the purified fluid sample will flow through the first channel, and, (e) detecting whether the purified fluid sample is diverted into said second channel or not, wherein diversion of the purified fluid sample into the second channel indicates the presence of the analyte of interest in the fluid sample and wherein the lack of diversion of the purified fluid sample into the second channel indicates the absence of the analyte of interest in the purified fluid sample. In some embodiments, the membrane is nanoporous. In some embodiments, the membrane is microporous. In some embodiments, the membrane is ultrathin. In some embodiments in which the membrane is ultrathin, the membrane is made of silicon, silicon nitride, silicon oxide, or silicon dioxide. In some embodiments, the ligand specifically binds said analyte. In some embodiments, the ligand is an antibody or antibody fragment retaining antigen-binding specificity. In some embodiments, the ligand non-specifically binds the analyte. In some embodiments, the ligand carries a charge that is opposite an overall charge on the analyte. In some embodiments, the analyte of interest is a virus. In some of these embodiments, the virus is SARS-CoV-2. In some embodiments, the analyte is a bacterium. In some embodiments, the analyte of interest is a protozoan. In some embodiments, the fluid sample is a patient sample. In some embodiments, the detection of whether the purified fluid sample is diverted into the second channel is by detecting movement of an indicator fluid or of a bubble. In some embodiments, the indicator fluid is an immiscible fluid. In some embodiments, the immiscible fluid is an oil. In some embodiments, the indicator fluid is colored. In some embodiments, the indicator fluid or bubble is a bubble. In some embodiments, the indicator fluid is an immiscible fluid. In some embodiments, the second channel has an exit port and the detection of whether the purified fluid sample is diverted into the second channel is by detecting fluid exiting the exit port.

In a third group of embodiments, the invention provides microfluidic devices for detecting an analyte of interest in a fluid sample, the device comprising: (a) a first microfluidic channel and a second microfluidic channel, each having a lumen defining a fluid path through the respective channels, the fluid path of each channel having a first end and a second end, wherein the first channel is positioned such that fluid sample will flow preferentially through the first channel in the absence of clogging of the fluid flow in the first channel, (b) a port for introducing the fluid sample into the first end of the first channel, (b) a nanoporous or microporous membrane disposed in the device such that the fluid sample must pass through the membrane to reach the second end of the first channel, and that, if the membrane becomes clogged, the fluid sample will be diverted instead into the first end of the second channel, (c) ligands that specifically or non-specifically bind the analyte of interest disposed on the membrane, whereby binding of the analyte of interest to the membrane clogs the membrane, (d) either (1) an indicator fluid or bubble disposed in second channel, wherein the indicator will move if the fluid sample is diverted into the second channel by the clogging of the membrane, or (2) wherein the second channel further has an exit port disposed at the second end of the second channel which allows fluid diverted into the first end of the second channel by blocking of the membrane to exit the channel, wherein movement of the indicator fluid or bubble, or exit of fluid from the exit port of the second channel, respectively, indicates the presence of the analyte of interest in the fluid sample. In some embodiments, the membrane is nanoporous. In some embodiments, the membrane is microporous. In some embodiments, the membrane is ultrathin. In some embodiments, the ultrathin membrane is made of silicon, silicon nitride, silicon oxide, or silicon dioxide. In some embodiments, the analyte of interest is a virus. In some embodiments, the virus is SARS-CoV-2. In some embodiments, the analyte is a bacterium. In some embodiments, the analyte of interest is a protozoan. In some embodiments, the fluid sample is a patient sample. In some embodiments, the indicator fluid or bubble is an immiscible fluid. In some embodiments, the indicator fluid is colored. In some embodiments, the immiscible fluid is an oil. In some embodiments, the oil is colored. In some embodiments, the said indicator fluid or bubble is a bubble. In some embodiments, the exit of the fluid from the exit port indicates the presence of the analyte of interest in the sample. In some embodiments, the ligand specifically binds said analyte. In some embodiments, the ligand is an antibody or an antibody fragment which retains antigen-binding specificity. In some embodiments, the ligand non-specifically binds the analyte. In some embodiments, the ligand carries a charge that is opposite an overall charge on the analyte.

### BRIEF DESCRIPTION OF THE DRAWINGS

FIGS. 1A and 1B. FIG. 1A. FIG. 1A shows a silicon chip holding with two “windows” exposing ultrathin membranes. FIG. 1B. FIG. 1B shows three hypothetical scenarios for interpreting the readout of indicator fluids of tests to determine the presence of a test pathogen, “COVID-19,” in samples. The left panel shows low levels of the indicator fluids in both the channel with an antibody binding the pathogen, and the reference channel, which does not. The low levels indicate that the pathogen was not detected in the sample.

FIG. 2. FIG. 2, left panel, shows a silicon chip bearing windows exposing ultrathin membranes for contact with fluid samples or control fluids. FIG. 2, middle panel, sets forth an exemplar method of fabricating microfluidic devices with an ultrathin membrane. FIG. 2, right panel, shows a commercially available spin-cup suitable for pre-filtering fluid samples.

FIGS. 3A-3D. FIGS. 3A-3D present a series of photographs showing use of one embodiment of a fluidic device of the invention. FIG. 3A. FIG. 3A shows the fluidic device, which contains an entry port at the top, two fluid paths, one of which passes through an ultrathin nanomembrane into a well, and an exit port for the second fluid path. FIG. 3B. FIG. 3B shows fluid being introduced from a pipette into the entry port disposed in the device on one side of the well. FIG. 3C. In FIG. 3C, fluid continues to be introduced from the pipette into the entry port as the well fills. FIG. 3D. FIG. 3D shows fluid coming out of the exit port of the second fluid path as the nanomembrane is clogged and the fluid is diverted into the second fluid path.

### DETAILED DESCRIPTION

Introduction

Ultrathin (<400 nm thick) precision pore membranes can be fabricated to be nanoporous or microporous, depending on the particular application for which they are to be employed. While ultrathin membranes have the benefit of being highly permeable, their use has been constrained by a significant drawback: they are easily clogged.

Surprisingly, the present invention turns the tendency of ultrathin membranes to clog, which has been considered a major disadvantage to their use, on its head. The present invention exploits the tendency of ultrathin membranes to clog by using that tendency to make them serve as fouling-sensitive hydraulic-switches in devices to detect whether a sample contains a virus or other analyte a practitioner wishes to detect. Even more surprisingly, the ultrathin membrane-based devices exploiting this discovery are inexpensive, rapid, require no external power to operate, and can be performed on samples available in only small volumes. Further, microfluidic devices employing ultrathin membranes as a fouling-sensitive hydraulic-switch can be multiplexed to test a sample for the presence of any of a number of analytes at the same time. Accordingly, the present invention represents a significant advance for detecting the presence of viruses and other analytes of interest in a sample.

The present invention was conceived during the COVID-19 pandemic in response to the need for a device that could provide a rapid and inexpensive test to determine if SARS-CoV-2 is present in a patient sample. The ultrathin membrane devices and methods can also, however, be used to detect the presence in a sample of any virus for which an antibody to a viral surface protein or other epitope has been developed. Moreover, the ultrathin membrane devices and methods can also be used to detect the presence in a sample of bacteria or of other analytes of interest, for which an antibody or portion of an antibody that retains its epitope-binding capability, is available that preferentially or specifically binds a surface epitope of the bacteria or other analyte of interest. Similarly, the inventive devices and methods can be used to detect the presence of analytes of interest using ligands other than antibodies that preferentially or specifically binds a characteristic particular to the analyte of interest. For example, if the practitioner wishes to determine whether a patient sample contains cells bearing a particular cytokine receptor on their surface, the cytokine can be attached to the ultrathin membrane in devices intended to detect the cells. Or, cells bearing angiotensin-converting enzyme 2 (ACE2) receptors can be disposed on the membrane to capture any SARS-CoV-2 viruses that may be present in a patient sample, as SARS-CoV-2 virus binds to that receptor. Other ligands that can be used to bind viruses, bacteria, cells, or other analytes of interest are known in the art, and it is expected that the practitioner can readily choose a ligand suitable to detect the presence of a particular analyte of interest to that practitioner.

Ultrathin membranes (sometimes referred to herein as “nanomembranes” or “membranes”) are available with pore sizes that make them either nanoporous (e.g., with pore diameters from 20 to 500 nm and with thicknesses from 20 to 400 nm) or microporous (e.g., with pores ranging in size from 500 nm to about 10 μm, and thicknesses of 100 nm to about 10 μm). Once the practitioner has selected a target organism or other analyte of interest, the practitioner selects an ultrathin membrane with a nanopore or micropore pore size that is larger than the target organism or other analyte of interest. In general, nanoporous membranes will be selected to capture viruses and other analytes of interest that have sizes comparable to that of normal viruses (as opposed to the uncommon Mimiviruses and other giant viruses), while microporous membranes will be selected to capture bacteria, protozoa, giant viruses, or eukaryotic cells of interest. It is expected that the person of skill can readily select a membrane pore size suitable to allow viruses or other analytes that are not being targeted for capture in the context of the use of the membrane whose pore size the practitioner is selecting, to flow through, while allowing targeted viruses or another analyte targeted by the practitioner to be bound by antibodies specific for the targeted virus or other targeted analyte. Any particular pore size can be readily tested to determine if it is appropriate for use with a particular analyte of a known size (such as a virus or a bacterium) by performing a test run with the analyte of interest, or with another analyte (such as another virus or bacterium) of a similar size, for which antibodies are conveniently available. After the antibodies for the analyte in the test run are disposed on the ultrathin membrane whose pore size is being tested, fluid containing the analyte bound by the antibodies is run over the membrane. If the membrane clogs, which can be determined by having the membrane disposed in one of the inventive devices and observing the indicator, the pore size is appropriate. If the membrane does not clog in the test run, as indicated by the flow switch not occurring, then the pores are too large and a membrane with smaller pores that do clog in the test run should be selected.

The ultrathin membrane selected can then be used in the inventive devices and methods to detect the presence of the selected target organism or other analyte of interest by, for example, coating or attaching to the ultrathin membrane antibodies that specifically bind a surface epitope on the target organism or other analyte of interest. In preferred embodiments, the surface epitope bound by the antibody is one that is not present on other organisms that might be expected to be present in the sample being tested and, in the most preferred embodiments, is an epitope that is unique to the target organism or other analyte of interest.

By selecting a nanoporous or microporous membrane with a pore size larger than the target organism or other analyte of interest, and an antibody or other ligand that specifically binds a surface epitope of the target organism or other analyte of interest, the inventive devices and methods can be used to detect the presence in a sample of one or more viruses, bacteria, protozoa, or eukaryotic cells of interest, such as circulating cancer cells. The viruses, bacteria, protozoa, and eukaryotic cells that can be detected using embodiments of the inventive devices and methods are sometimes collectively referred to herein as “analytes of interest.” As used herein, the terms “targeted virus,” “targeted bacteria,” “targeted protozoa,” or “targeted cell,” refer to the particular organisms or cells of the stated type the practitioner is trying to detect using the inventive devices and methods.

The sample may be in various forms known in the art. In medical uses, it may be, for example, a cheek or nasal swab, saliva, a blood sample, or a tissue biopsy. In the case of swabs or biopsies, the swab or biopsy tissue is typically contacted with a fluid such as sterile saline to transfer to the fluid pathogens that may be present in the swab or biopsied tissue. It is assumed persons of skill are familiar with such procedures. In a preferred group of embodiments, the sample is from a subject who is being screened for the presence of a pathogen, such as SARS-CoV-2. In non-medical embodiments, the sample may be, for example, a swab of a solid food, such as chicken or ground meat, to monitor whether the food contains Salmonella or other pathogens. In yet other embodiments, the sample may be an environmental sample, such as water from a lake, a beach, or a reservoir, to detect the presence of E. coli or giardia. The samples are typically prepared for use in the inventive devices and methods by removing debris and other materials that are larger than the size of the pores of the ultrathin membranes before the purified sample is provided to the inventive devices. Methods for pre-filtering samples to remove debris and other contaminants that will block flow through the ultrathin membranes are discussed in more detail below.

In some embodiments, the devices and methods provide multiplex detection, that is, detection of whether two or more target viruses or other analytes of interest are present in a sample using a single device in a single workflow. Further, in some embodiments, the devices are sized to fit into a standard multi-well plate, permitting the devices to be used in robotic or other automated handling applications. In one embodiment, the devices are configured to fit over two wells of a 96-well plate, allowing 48 tests to be run at a time in an automated system.

The inventive devices and methods exploit the tendency of ultrathin membranes to clog, previously seen as a liability, to provide an advantage that allows detecting the presence of an analyte of interest, such as SARS-CoV-2, in a sample. The high permeability of ultrathin membranes, combined with the tendency to clog, allows the membranes to be used as a novel transduction principle in which a nanomembrane is used as a fouling-sensitive hydraulic switch. The membranes are designed to selectively clog from being contacted with SARS-CoV-2 (or with another analyte which they are designed to detect), but nothing else likely to be in the sample. For convenience of reference, the discussion below will generally be couched in terms of the analyte of interest being a virus, unless otherwise indicated. The same discussion will generally pertain to bacteria, protozoa, or eukaryotic cells of interest, with appropriate adjustment of the size of the pores of the nanomembrane or microporous membrane.

In some embodiments of the inventive devices and methods, the device is a microfluidic device. Conveniently, the device is a silicon chip bearing an ultrathin membrane. Ultrathin membrane fouling is converted into a detector by using a first set comprising a first microfluidic flow path, or “first channel”, and a second microfluidic flow path, or “second channel.” The first channel has a first end and a second end, such that fluid can flow, except as described below, between the first end of the first channel and the second end of the first channel. The first channel contains within it between the first end and the second end an ultrathin microporous or nanoporous membrane, which membrane is disposed within the channel such that fluid cannot pass through the channel from the first end to the second end within passing through the pores in the membrane. The ultrathin membrane has pore walls that are coated with or linked to a ligand, such as an antibody, that binds specifically to a surface feature of the analyte of interest. For example, if the analyte of interest is a virus, and the ligand to be used to capture it is an antibody, the antibody chosen will be one that specifically binds an epitope present on the surface of the virus. In some embodiments, the antibody or other ligand of interest is covalently bound to the walls of the pores. The first channel has a port at the first end. The port allows the introduction of a fluid sample into the first end of the channel.

As noted, the microfluidic device further comprises a second channel, which second channel has first end and a second end, which first end and said second are fluidly connected. The first end of the second channel has an opening fluidly connected to the first channel and disposed between the first end of the first channel and the membrane. The opening of the first end of the second channel is positioned, configured, or both, so that fluid will not flow into and through the first end of the second channel to the second end of the second channel unless the membrane in the first channel becomes clogged, thereby increasing the resistance of the membrane to fluid flow to exceed the resistance to fluid flow of the second channel For example, in some embodiments, the second channel is disposed above the first channel, so that gravity will keep the fluid sample from entering the first end of the second channel unless the resistance of the membrane increases due to clogging. Alternatively, the entrance of the first end of the second channel is smaller than the diameter of the lumen of the first channel, such that the hydraulic resistance of the first channel is lower than that of the second channel in the absence of clogging of the membrane.

In some preferred embodiments, both flow paths have hydraulic resistances so low that fluid can be driven by the surface tension of a sample applied to an upstream port. In other embodiments, fluid in the sample can be driven through the membrane (until and unless it is clogged by binding of the analyte of interest to ligand on the membrane) by pressure applied by a plunger or other conventional method of applying hydraulic force to introduce samples into a microfluidic device.

Preferably, the epitope chosen as the target is one unique to that virus or other analyte of interest the presence or absence of which in the sample the practitioner is trying to determine. For example, if the analyte of interest is the virus SARS-CoV2, the antibody chosen by the practitioner is preferably one that specifically binds the so-called “spike” protein of SARS-CoV2. The pores in the ultrathin membrane are selected to be of a size so that viruses will go through the membrane unless they contact and are bound by the antibody. If virus recognized by the antibody is present in the sample, it will the antibody on the walls of the pores of the membrane and rapidly clog the pores of the membrane and change it from being highly permeable to being highly resistive. This change in permeability due to the clogging of the first channel shunts the flow to the second channel. Typically, the second end of the second channel is provided with an exit.

Flow of the fluid into and through the second channel can be detected, which indicates that the analyte of interest is present in the sample. In some embodiments, the user detects the diversion of fluid into the second channel due to a clogged membrane by simply observing the presence of a drop of fluid or bubbling at the exit of the second channel, either by eye or by an automated reader. In other embodiments, the second channel can have disposed within it an indicator allowing visualization of fluid movement in the second channel. In these embodiments, movement of the indicator within the second channel signals that the ultrathin membrane has been clogged and that the analyte of interest is therefore present in the sample.

In some embodiments with an indicator, the indicator is a fluid immiscible in the fluid of the fluid sample. As the fluid sample will usually be water-based, conveniently, the immiscible indicator fluid is an oil. The indicator fluid is preferably colored, which allows easier detection of its displacement by providing a visual signal that the analyte of interest is present. In some embodiments, the indicator is a bubble, such as an air bubble, which can be in oil. The movement of the indicator fluid or bubble can be read by eye (either directly or under magnification), or by an automated reader. Fluid motion can be detected by, for example, having a photoelectric cell detect if light passing through a window in the device is obstructed by indicator fluid or fluid exiting the second exit rising above a set point which indicates the fluid has been displaced. Air bubble movement can also be detected by a laser passing though the fluid channel. If the indicator is a bubble, and the bubble enters the light path, it scatters the light, and the machine records presence of the analyte of interest. If the antibody or other ligand does not capture the analyte of interest, flow passes through the nanomembrane in the first channel and the indicator fluid in the second channel does not move. For ultrathin membranes, the operation requires no external electrical power: capillarity alone, or the pressure from pipetting fluids into the device can be used to power the flow, the filtration, and the membrane switch.

The sharpness and location of this ‘switch’ in permeability is tunable by the practitioner to match the properties for the target analyte of interest by matching the membrane properties (pore size and density, membrane area) to solute properties (size and concentration). By contrast, conventional polymer membranes (˜10 μm thick) are: 1) too resistive for use with low pressure microfluidics, and 2) insensitive to particle capture because of a high loading capacity.

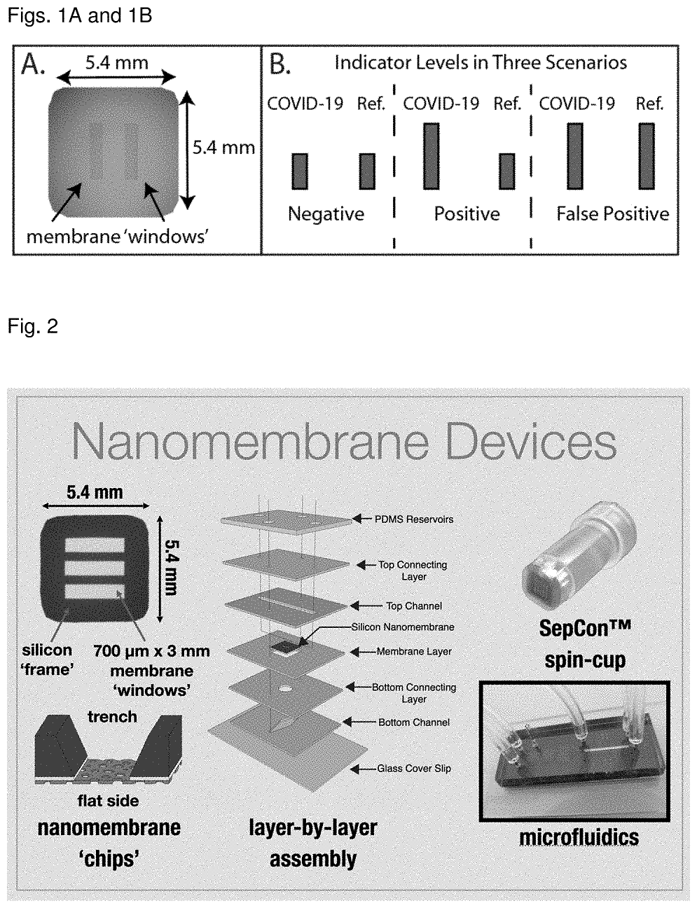

In some embodiments, a second set of two microfluidic flow paths with an ultrathin, nanoporous membrane is provided to provide an internal control to detect false positives. An exemplar dual nanoporous membrane silicon chip is shown in FIG. 1A. The nanomembranes are held by the chip, but accessible through the so-called “windows.” A reference sample, typically containing a virus that is not specifically bound by the antibody being used to capture the analyte of interest, or of just carrier fluid, is flowed through the second nanomembrane. Referring to FIG. 1B, the levels of the indicator fluid reflect the three possible scenarios. The left panel of the Figure shows two equal, low levels, indicating, in this hypothetical, that the SARS-CoV2 virus (“COVID-19”) was not detected in the sample. In the middle panel of the Figure, the left indicator level (labeled “COVID-19”) is high, and the right level, for the internal control is low, showing a detection of SARS-CoV2 virus in the sample. In the right panel, both the left and the right indicator levels are high, showing a false positive.

FIG. 2 depicts the construction of a microfluidic device containing top and bottom channels with a nanomembrane disposed between the channels.

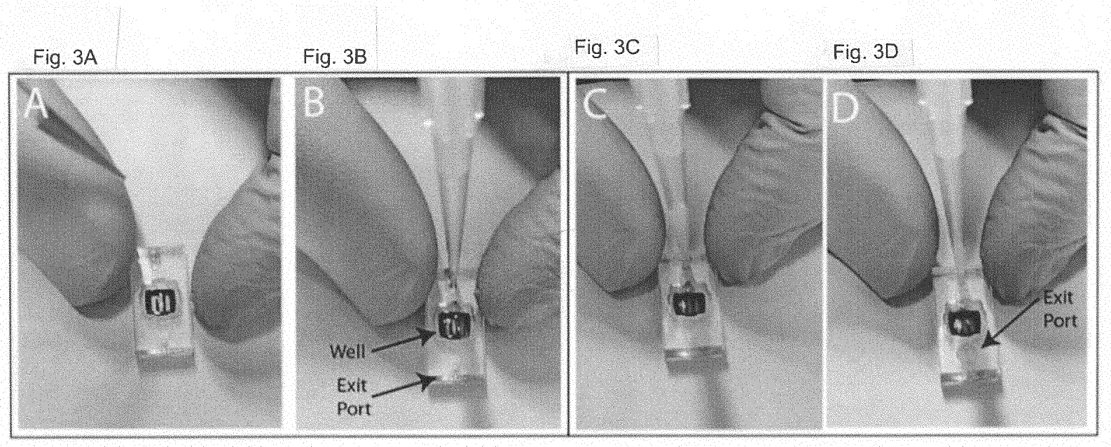

FIGS. 3A-D show testing of an embodiment of a fluidic device containing two fluid paths, one of which passes through an ultrathin membrane into a well (FIG. 3A). In FIG. 3B, fluid is introduced into an entry port disposed in the device above the well. In FIG. 3C, fluid continues to be introduced into the entry port as the well fills. FIG. 3D shows fluid coming out of the exit port of the device as the membrane is blocked and the fluid is diverted into the second path.

Multiplexing

In some embodiments, the devices are configured for multiplex testing, that is, for testing for the presence of multiple analytes, or of multiple samples at the same time. In some of these embodiments, the devices can be used to detect the presence of more than one analyte in the sample by coating it with, or attaching to it, antibodies against more than one virus or other analyte of interest. A positive result (such as by bubbling at the exit of the second channel or displacement of an indicator fluid or a bubble in the second channel) means that at least one of the analytes to which one of the antibodies on the membrane is present in the sample. Alternatively, the device can be configured for multiplexing by having a plurality of membranes, preferably with an “x+1” configuration, in which x represents two or more membranes, each of which bears antibodies to detect a different analyte, such as a particular viral or bacterial species, and the “+” represents a membrane to provide an internal control, as discussed in the preceding section. In some of these embodiments, the viruses or other analytes of interest chosen for detection are grouped according to the type of sample in which they might be found. For example, antibodies for different respiratory viruses or bacteria might be on one multiplex device, while antibodies for viruses or bacteria that might be present in a blood sample might be included on a second multiplexed device, allowing the practitioner to provide the sample to a device that can screen the sample for a number of viruses that might be of interest in the sample.

Ultrathin Membranes

Ultrathin (<400 nm thick) precision pore membranes have been made and their properties explored, as exemplified by Striemer, et al., Nature, 2007. 445(7129): p. 749-753; DesOrmeaux, et al., Nanoscale, 2014. 6(18): p. 10798-10805; and Winans, et al., J Memb Sci, 2016. 499: p. 282-289. Ultrathin membranes exhibit a unique combination of filtration properties. First, they are exceptionally permeable, enabling very low pressure filtration in microfluidic devices. Second, they clog rapidly if the size and number of nanoparticle solutes are matched to the pore size and density of the membrane. This tendency to clog rapidly has limited the ability of the membranes to process large volumes or very concentrated small volumes, but has made them useful in processing dilute samples.

Ultrathin membranes have been made using pure silicon, silicon nitride, glass (SiO₂), MgFl₂, gold, graphene, and various polymers. Because of their extreme thinness, ultrathin membranes are sometimes referred to as “2D membranes.” It is contemplated that ultrathin membranes made of any of the materials mentioned above can be used in the inventive devices and methods. In preferred embodiments, the ultrathin membranes are made of silicon, silicon nitride, silicon oxide, or silicon dioxide, as ultrathin membranes made of these materials are particularly robust.

Silcon nanoporous and microporous membranes suitable for use in the inventive devices are commercially available from SiMPore Inc. (West Henrietta, N.Y.) and Aquamarijn Micro Filtration BV (Zutphen, The Netherlands). SiMPore's website states that it provides nanoporous silicon and silicon nitride membranes with tunable pore distribution and porosity and membrane areas from less than 10×10 μm to more than 0.5×5 mm The website further indicates that it provides microporous silicon oxide membranes and microporous silicon nitride membranes, with membrane areas from less than 10×10 μm to more than 2 mm×2 mm

Use of Thicker Membranes

Ultrathin membranes are particularly preferred embodiments of the inventive devices and methods, in part because their thinness allows fluid to flow through the membrane by capillarity or the pressure provided by pipetting the fluid onto the membrane. The principle of exploiting the fouling of a membrane to detect the presence of an analyte binding to ligands can, however, also be exploited in the context of conventional, non-ultrathin, membranes that have nanopores or micropores.

Membranes of conventional thickness provide more resistance to fluid flow than do ultrathin membranes. Embodiments using membranes that are not ultrathin, therefore, will typically include a source of pressure, or pressure gradient to push or to pull fluid through the membrane. For example, the fluid being introduced onto the membrane can be under pressure provided by a syringe-like arrangement, in which a plunger exerts force assisting the flow of the fluid through the membrane until it becomes sufficiently fouled so as to divert the fluid flow into a second fluid path.

Attaching Antibodies or Other Ligands

Attaching antibodies to or coating silicon membranes with antibodies or other ligands that can specifically bind to an analyte of interest, can be accomplished by conventional methods known in the art. Preferably, the antibodies are attached by chemical techniques for functionalizing a silicon substrate. For example, maleimide—thiol chemistry can be used to link antibodies to the silicon membrane, as can silanes. The Examples set forth a method using streptavidin-biotin to couple biotinylated antibodies to the membranes. In some embodiments, the nanomembrane may simply be dipped in a solution containing the antibodies and allowed to dry. Some antibodies are expected to remain on the membrane by physical adsorption. While intact immunoglobulins can be used, a variety of antibody fragments that retain antigen binding are known and can be used at the practitioner's discretion.

In most embodiments, ligand specific for the analytes of interest are contemplated. In some limited circumstances, however, a non-specific ligand can be used. For example, if the virus or other analyte of interest is known to be charged positively or negatively charged, and a relatively purified solution containing the analyte is available, the membrane can be functionalized with a material that carries the opposite charge to capture the analyte by electrostatic interaction rather than specific binding. For example, if the analyte is negatively charged, the membrane can be functionalized with groups bearing positive charges. A preferred method of functionalizing membranes is the use of KODE™ amphiphilic coating molecules (Kode Biotech Ltd., Auckland, New Zealand), discussed in more detail in Example 1. A number of suitable specific and non-specific ligands are known in the art for particular types of analyte of interest (e.g., viruses, bacteria, protozoans, cells from a patient sample, such as circulating cancer cells), and it is believed that the person of skill in the art can choose a particular ligand suitable to bind to or capture particulars analyte of interest.

Sample Preparation

As noted above, one of the characteristics of ultrathin membranes is their tendency to clog easily, a feature referred to more formally as “low binding capacity.” The tendency to clog easily means the ultrathin membranes could be easily clogged if the sample being tested contains debris of the size of the ultrathin membrane's pores or larger. Samples are therefore preferably subjected to filtration or other methods known in the art to remove debris, cellular components, or other material in the sample that is larger than the size of the pores to reduce the chance they will be clogged with contaminants before they can capture any analyte that might be present in the sample. Conveniently, this can be accomplished by placing a fluid sample in a container in which fluid cannot go from the entrance to a holding area without passing through a nanomembrane positioned between the entrance and the holding area to filter the fluid sample before it is provided to the inventive devices for analyte detection. The “pre-filter” membrane differs from the one used for analyte detection in particular in that it does not have a ligand, such as an antibody, on the membrane that would bind the analyte of interest, and preferably does not have any ligand on it, so that it serves only to filter contaminants that would non-specifically clog the membrane used for analyte detection.

A commercially available container with such a nanomembrane, a SepCon™ spin-cup, is shown in FIG. 2. The container is preferably configured to fit into a microfuge or other low-G centrifuge so that centrifugation will assist forcing the fluid through the nanomembrane, leaving debris and other large contaminants behind. The fluid in the holding area can then held in the holding area (with the container preferably capped to avoid sample loss through evaporation) until it is be transferred to one of the inventive devices to be tested for the presence of the analyte of interest. After this pre-filtering step, the only materials entering the device will be smaller than the pores. The materials will then only clog the membrane through affinity interactions with antibodies or other capture molecules disposed on or around the membrane pores.

Avoiding or Reducing False Positives

Engineering diagnostics to minimize false positives limits hospital burden and patient stress. Embodiments of the inventive devices include several intrinsic and engineered features that help reduce the rate of false positives: 1) The flow focuses particles to the center of pores so that wall binding is unlikely to occur without affinity capture by the antibody. 2) In some embodiments, the devices include a second, reference membrane, bearing a non-specific, control antibody. The two membranes can be individually coated on the same chip with a S3 piezo-droplet device (SCIENION US Inc., Tempe, Ariz.). If the reference channel is positive, a factor other than COVID-19 is responsible and the result is read as a false positive; 3) Patient samples can be pre-cleared by filtration of a larger sample (˜200 μL) through a membrane with the same size pores as the sensor membrane, but without any affinity coating. In this way, all materials that enter the device are too small to physically clog the pores. The fouling switch is then activated only by affinity-based binding to the pore walls. 4) Membranes are robust at the low pressures associated with passive operation. In the event of breakage or other membrane failure, the device will produce a negative result.

### EXAMPLES

### Example 1

This Example describes materials and methods for an exemplar embodiment of the invention.

Nanoparticle Surrogates for SARS-CoV-2: Nanoparticles are coated with the surface ‘spike’ proteins nCoV-S1 and nCoV-S2, which are commercially available as recombinant proteins from New England Peptide Inc. (Gardner, Mass.). These large glycoproteins are responsible for initial SARS-CoV-2 attachment to cells. The proteins are biotinylated with commercial kits and anchored to 120 nm strepavidin coated silica beads (Corpuscular Inc., Cold Spring, N.Y.) as models of the virus. Non-SARS-CoV-2 spike proteins (MERS; HKU1 peptides (MyBiosource, Inc., San Diego, Calif.), are used as control surrogates.

Membrane Coatings: Antibodies are adhered to membrane surfaces using KODE™ amphiphilic coating molecules (Kode Biotech Ltd., Auckland, New Zealand). KODE™ molecules conformally coat membranes in a thin (<100 nm) layer with a simple dip coating and are stable under flow. KODE-biotin is applied, followed by strepavidin, and a 50/50 mixture of both spike protein antibodies in a biotinlyated form (MyBiosource) is applied. To prevent nanoparticles coated with viral protein from binding to pore walls through avidin-biotin affinity rather than antibody-antigen affinity, blocking solutions are used on the membranes at various stringencies until there is a clear difference in the capture of spike-coated vs. uncoated particles. Alternatively, the antibodies are coupled to membranes using KODE™-maleimide with thiol linker chemistry, by using protein G as an intermediate, or both.

Sensitivity: The size and number of pores in the membranes is tuned to achieve: 1) low pressure baseline operation, so that the membrane path is the lowest resistance path in the absence of viral surrogates; and 2) a switching like mechanism when viral surrogates are present. Membranes are produced at two different sensitivities to illustrate the ability to tune the sensor switch to detect both 10⁴ and 10⁷ particles/mL. This is the range of viral loads seen with COVID-19 in early reports from patient samples. Image analysis of confocal images and electron microscopy is used to verify that nanopartcles coated with viral proteins are caught in nanomembranes at the expected density.

### Example 2

This Example describes an exemplar device of the invention.

An exemplar microfluidic device to detect the presence of an analyte of interest has a microfluidic channel across the top of the device. A sample port on the first end of the microfluidic channel. Half-way down the channel is an ultrathin nanoporous membrane bearing antibodies which specifically bind the analyte of interest. The membrane covers the opening into a second microfluidic channel which, like a sewer drain, is positioned below the top channel. The fluid sample will therefore flow into the second channel as long as the pores of the membrane are open. If the membrane becomes clogged upon addition of the fluid sample due to the binding of the analyte to the antibodies, the fluid sample will continue instead along the top channel to its end, where it contacts and displaces an indicator, such as an indicator fluid, signaling blockage of the lower resistance channel, and the consequent detection of the analyte. Before the sample is introduced to the device, the device is prefilled (either at the time of manufacture or at or shortly before use) with saline solution, which will be displaced by the sample when it is added. The saline solution provides fluid on both sides of the membrane and removes the possibility that surface tension will interfere with passage of the sample through the filter.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A microfluidic device for detecting an analyte of interest in a fluid sample, said device comprising:
(a) a first microfluidic channel and a second microfluidic channel, each having a lumen defining a fluid path through said channel, said fluid path of each channel having a first end and a second end, wherein said first channel is positioned such that fluid sample will flow preferentially through said first channel in the absence of clogging of said fluid flow in said first channel,
(b) a port for introducing said fluid sample, said port being fluidly connected to said first end of said first channel,
(c) a nanoporous or microporous membrane disposed in said device such that said fluid sample must pass through said membrane to reach said second end of said first channel, and that, if said membrane becomes clogged, said fluid sample will be diverted instead into said first end of said second channel,
(d) ligands that specifically or non-specifically bind said analyte of interest disposed on said membrane, whereby binding of said analyte of interest to said membrane clogs said membrane,
(e) either (1) an indicator fluid or bubble disposed in second channel, wherein said indicator will move if said fluid sample is diverted into said second channel by said clogging of said membrane, or (2) wherein said second channel further has an exit port disposed at said second end of said second channel which allows fluid diverted into said first end of said second channel by blocking of said membrane to exit said channel, wherein movement of the indicator fluid or bubble, or exit of fluid from said exit port of said second channel, respectively, indicates the detection of said analyte of interest in said fluid sample.

2. The microfluidic device of claim 1, wherein said membrane is nanoporous.

3. The microfluidic device of claim 1, wherein said membrane is ultrathin.

4. The microfluidic device of claim 3, wherein said ultrathin membrane is made of silicon, silicon nitride, silicon oxide, or silicon dioxide.

5. The microfluidic device of claim 1, wherein said analyte of interest is a virus, a bacterium, a protozoan, or a eukaryotic cell.

6. The microfluidic device of claim 1, wherein said indicator fluid or bubble is an immiscible fluid, optionally wherein said immiscible fluid is a colored oil.

7. The microfluidic device of claim 1, wherein said ligand an antibody or an antibody fragment retaining antigen-binding specificity.

8. A method for detecting whether an analyte of interest is present in a fluid sample, said analyte having a size, said method comprising:
(a) selecting a first nanoporous or microporous membrane with pore sizes larger than said size of said analyte,
(b) purifying said fluid sample to remove debris that is larger than said size of said analyte, by passing said fluid sample through said first nanoporous or microporous membrane, thereby obtaining a purified fluid sample,
(c) introducing said purified fluid sample to a port of a first channel of a microfluidic device, thereby flowing said purified fluid sample into said first channel,
(d) contacting said purified fluid sample to a second nanoporous or microporous membrane, which second nanoporous or microporous membrane is disposed in said first channel, and which bears ligands that specifically or non-specifically bind said analyte of interest to said second nanoporous or microporous membrane, whereby binding of said analyte of interest to said second nanoporous or microporous membrane will clog said second nanoporous or microporous membrane and divert said purified fluid sample into a second channel, and wherein if said analyte of interest is not present, said purified fluid sample will flow preferentially through said first channel, and,
(e) detecting whether said purified fluid sample is diverted into said second channel, wherein diversion of said purified fluid sample into said second channel indicates the presence of said analyte of interest in said fluid sample and wherein said lack of diversion said purified fluid sample into said second channel indicates the absence of said analyte of interest in said purified fluid sample.

9. The method of claim 8, wherein said either of said first membrane and said second membrane are nanoporous.

10. The method of claim 8, wherein said either of said first membrane and said second membrane is microporous.

11. The method of claim 8, wherein said second membrane is ultrathin and is made of silicon, silicon nitride, silicon oxide, or silicon dioxide.

12. The method of claim 8, wherein said ligand is an antibody or an antibody fragment that retains antigen-binding specificity.

13. The method of claim 8, wherein said analyte of interest is a virus, a bacterium, a protozoan, or a eukaryotic cell.

14. The method of claim 8, wherein said detection of whether said purified fluid sample is diverted into said second channel is by detecting movement of an indicator fluid or of a bubble.

15. The method of claim 8, wherein said second channel has an exit port and said detection of whether said purified fluid sample is diverted into said second channel is by detecting fluid exiting said exit port.

16. A microfluidic device for detecting an analyte of interest in a fluid sample, said device comprising:
(a) a first microfluidic channel and a second microfluidic channel, each having a lumen defining a fluid path through said channel, said fluid path of each channel having a first end and a second end, wherein said first channel is positioned such that fluid sample will flow preferentially through said first channel in the absence of clogging of said fluid flow in said first channel,
(b) a port for introducing said fluid sample, said port being fluidly connected to said first end of said first channel,
(c) a nanoporous or microporous membrane disposed in said device such that said fluid sample must pass through said membrane to reach said second end of said first channel, and that, if said membrane becomes clogged, said fluid sample will be diverted instead into said first end of said second channel,
(d) ligands that specifically or non-specifically bind said analyte of interest disposed on said membrane, whereby binding of said analyte of interest to said membrane clogs said membrane,
(e) either (1) an indicator fluid or bubble disposed in second channel, wherein said indicator will move if said fluid sample is diverted into said second channel by said clogging of said membrane, or (2) wherein said second channel further has an exit port disposed at said second end of said second channel which allows fluid diverted into said first end of said second channel by blocking of said membrane to exit said channel, wherein movement of the indicator fluid or bubble, or exit of fluid from said exit port of said second channel, respectively, indicates the presence of said analyte of interest in said fluid sample.

17. The microfluidic device of claim 16, wherein said membrane is nanoporous.

18. The microfluidic device of claim 16, wherein said membrane is microporous.

19. The microfluidic device of claim 16, wherein said membrane is ultrathin and is made of silicon, silicon nitride, silicon oxide, or silicon dioxide.

20. The microfluidic device of claim 16, wherein said ligand is an antibody or an antibody fragment that retains antigen-binding specificity.
